(19) United States
(12) Patent Application Publication
(10) Pub. No.: US 2014/0065202 A1
(43) Pub. Date: Mar. 6, 2014

(54) COMPOSITE FILM

(71) Applicant: FUJIFILM Corporation, Tokyo (JP)

(72) Inventor: Koju ITO, Kanagawa (JP)

(73) Assignee: FUJIFILM Corporation, Tokyo (JP)

(21) Appl. No.: 14/075,614

(22) Filed: Nov. 8, 2013

Related U.S. Application Data
(63) Continuation of application No. PCT/JP2012/060319, filed on Apr. 17, 2012.

(30) Foreign Application Priority Data
May 11, 2011 (JP) 2011-105887

(51) Int. Cl. A61K 9/70

(52) U.S. Cl. CPC A61K 9/7007
USPC 424/443

(57) ABSTRACT

A composite film comprises a drug sustained-release portion and an adhesive portion which are layered on each other. The drug sustained-release portion has film-like drug layers and a film-like biodegradable polymer layer. The drug sustained-release portion has the drug layer, the biodegradable polymer layer, and the drug layer in this order from the adhesive portion side. Each drug layer is a drug form containing a drug of a predetermined concentration or more. The drug is released from the drug layer exposed. When the drug layer disappears, a biodegradable polymer of the biodegradable polymer layer starts to degrade and/or dissolve. When the biodegradable polymer layer disappears, the drug is released from the drug layer adjoining the adhesive portion. A plurality of pores are formed in the adhesive portion. The pores retain water due to capillary force.

### CROSS REFERENCE TO RELATED APPLICATIONS

This application is a continuation of PCT/JP2012/060319 filed on Apr. 17, 2012, which claims foreign priority to Japanese Application No. 2011-105887 filed on May 11, 2011. The entire contents of each of the above applications are hereby incorporated by reference.

### BACKGROUND OF THE INVENTION

1. Field of the invention

The present invention relates to a composite film having drug sustained-release characteristics and adhesiveness.

2. Description Related to the Prior Art

A drug agent such as an anti-cancer agent is given to a living body through blood, by mouth, or the like. It is necessary to give a high dose of the drug agent to the living body to make a drug in the drug agent surely reach an intended site, for example, an affected area, because the drug is also absorbed by sites other than the intended site. The high dose of the drug agent may cause problems such as side-effects and an overdose resulting in poor efficacy.

One of the methods to solve the above-described problems is to directly attach a film containing a drug to the affected area of the living body, for example. An example of such is a honeycomb-patterned porous structure disclosed in Japanese Patent Laid-Open Publication No. 2007-061559. The honeycomb-patterned porous structure carries a drug or a drug form, which contains the drug, dispersed therein. The honeycomb-patterned porous structure is indwelled or placed in the body through laparotomy or the like. When the honeycomb-patterned porous structure is made from a biodegradable material as disclosed in the Japanese Patent Laid-Open Publication No. 2007-061559, it degrades or dissolves naturally within a period of time after being indwelled. There is no need to take it out by a reoperation. Thus the use of the biodegradable material is advantageous in reducing physical stress on the living body.

The film requires an adhesive function to be adhered to a predetermined site inside the body. Examples of such adhesive films include honeycomb-patterned films disclosed in, for example, WO2006/022358, Japanese Patent Laid-Open Publication Nos. 2007-204524 and 2008-012216.

An amount of a drug appropriate for treatment falls within a certain range and this range is called a therapeutic range. An amount of a drug exceeding the therapeutic range may produce side-effects and even reach a lethal dose. The drug may be ineffective when the amount is less than the therapeutic range. The honeycomb-patterned porous structure disclosed in the Japanese Patent Laid-Open Publication No. 2007-061559 may possibly be indwelled in the body and the drug may possibly be supplied to an intended site, but the honeycomb-patterned porous structure may degrade or dissolve too fast. In other words, at an initial stage after indwelling, a drug release amount may exceed the therapeutic range drastically. Thereafter, the drug release amount may be less than the therapeutic range. In some cases, the honeycomb-patterned porous structure may run out of the drug to be given. Hence, the honeycomb-patterned porous structure disclosed in the Japanese Patent Laid-Open Publication No. 2007-061559 has not achieved a level to allow estimation of whether the drug release amount is within the therapeutic range.

The honeycomb-patterned films disclosed in the WO 2006/022358 and the Japanese Patent Laid-Open Publication Nos. 2007-204524 and 2008-012216 may have certain effects in view of indwelling in the body, but they have not achieved the reliable drug delivery and have not touched upon the therapeutic ranges of the drugs.

### SUMMARY OF THE INVENTION

An object of the present invention is to provide a composite film which releases a drug at an intended site, with a predetermined drug release amount within a predetermined range for a long period of time.

In order to achieve the above and other objects, the composite film of the present invention comprises a drug sustained-release portion and an adhesive portion. The drug sustained-release portion contains a biodegradable polymer and a drug form. The drug form contains a drug of a predetermined concentration or more. The drug sustained-release portion releases the drug from the drug form through at least one of degradation and dissolution of the biodegradable polymer. The drug sustained-release portion is adhered to a target site through the adhesive portion. The adhesive portion is layered on the drug sustained-release portion.

It is preferable that the drug sustained-release portion has (A) or (B):

(A) the film-like drug form and the film-like biodegradable polymer on the film-like drug form;

(B) the particle-like drug forms and the biodegradable polymer in which the particle-like drug forms are embedded.

It is preferable that a plurality of pores are formed into an array on one surface of the adhesive portion, opposite to the other surface in contact with the drug sustained-release portion. It is preferable that the pores of the adhesive portion retain water of the target site due to capillary force and thereby the drug sustained-release portion is adhered to the target site through the adhesive portion.

It is preferable that the adhesive portion is composed of a biodegradable polymer in which at least one of a degradation rate and a dissolution rate is smaller than that of the biodegradable polymer of the drug sustained-release portion.

According to the composite film of the present invention, the drug is released at an intended site, with a drug release amount within a predetermined range for a long period of time.

### BRIEF DESCRIPTION OF THE DRAWINGS

The above and other objects and advantages of the present invention will be more apparent from the following detailed description of the preferred embodiments when read in connection with the accompanied drawings, wherein like reference numerals designate like or corresponding parts throughout the several views, and wherein:

FIG. 1 is a cross-sectional view of a composite film of the present invention;

FIG. 2 is a graph representing a relation between a drug release amount and time: a curve A depicted by a solid line corresponds to the composite film of the present intention; a curve B depicted by a chain double-dashed line corresponds to a conventional film;

FIG. 3 is a cross-sectional view of a composite film of the present invention;

FIG. 4 is a cross-sectional view of a drug particle;

FIG. 5 is a plan view of a composite film of the present invention;

FIG. 6 is a cross-sectional view along a line VI-VI of FIG. 5;

FIG. 7 is a cross-sectional view along a line VII-VII of FIG. 5;

FIG. 8 is a cross-sectional view of a composite film of the present invention;

FIG. 9 is a cross-sectional view along another line across the composite film in FIG. 8;

FIG. 10 is a cross-sectional view of a composite film of the present invention; and

FIG. 11 is a perspective view of a composite film of the present invention.

### DESCRIPTION OF THE PREFERRED EMBODIMENTS

A composite film of the present invention is a drug sustained-release film with a drug sustained-release function. The drug sustained-release function is to gradually release a drug to the outside of the composite film. The composite film also functions as an adhesive film which is attached to an intended site or a target site. The composite film of the present invention is designed to be attached to an inner surface or an outer surface (or skin) of a living body. For example, the composite film is attached to a target site, for example, an affected area located on the inner surface or the skin and stays where it is attached. Thereby, the composite film releases the drug gradually at or around the target site.

Hereinafter, embodiments of the composite film of the present invention are described.

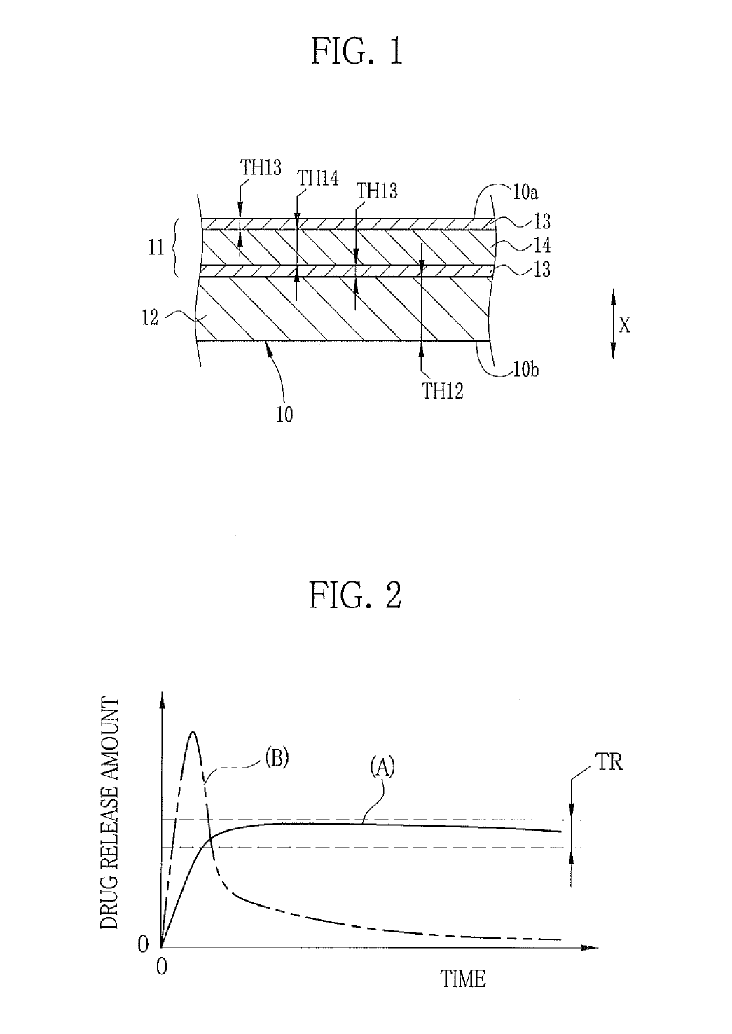

A composite film 10 of a first embodiment comprises a drug sustained-release portion 11 and an adhesive portion 12. The drug sustained-release portion 11 releases a drug gradually. The drug sustained-release portion 11 is adhered to a target site through the adhesive portion 12. The target site is an object or a site or apart of the object to which the drug sustained-release portion 11 or one of drug sustained-release portions of the following embodiments is adhered. For example, when the target site is a site of a living body, the drug sustained-release portion 11 is adhered to the target site through the adhesive portion 12. The adhesive portions of the following embodiments act in a similar manner. The drug sustained-release portion 11 and the adhesive portion 12 are layered on each other in the thickness direction indicated by an arrow X. In FIG. 1, “10a” denotes a film surface of the drug sustained-release portion 11, and “10b” denotes a film surface of the adhesive portion 12.

The drug sustained-release portion 11 has a film-like drug layer 13 and a film-like biodegradable polymer layer 14. The drug layer 13 is a drug form that contains a drug of a predetermined concentration or more. The drug form may consist solely of drug(s). Thus, the drug sustained-release portion 11 includes the drug form and the biodegradable polymer.

Generally, the biodegradable polymer has hydrolyzability and bioabsorbability in addition to biodegradability, meaning that the polymer is degraded by microorganisms. In the present invention, however, the biodegradable polymers in the biodegradable polymer layers 14 described in the following embodiments are not used for their biodegradability caused by the microorganisms. Instead, they are used for their degradability (for example, hydrolyzability) other than the biodegradability, water-solubility, absorbability to water, or the like. Hence, a water-soluble polymer or a hydrolyzable polymer may be used instead of or in addition to the biodegradable polymer.

The film-like drug layer 13 and the film-like biodegradable polymer layer 14 are layered or stacked on each other in the thickness direction X. For example, as shown in FIG. 1, the drug sustained-release portion 11 of the composite film 10 is composed of three layers: the drug layer 13, the biodegradable polymer layer 14, and the drug layer 13, layered in this order on the adhesive portion 12. Note that the two drug layers 13 may contain different drugs.

Owing to the above structure, the composite film 10 starts releasing the drug from the drug layer 13 through the film surface 10a. The thickness of the drug layer 13 gradually decreases with the release of the drug, and the drug layer 13 disappears eventually. Thereby, the biodegradable polymer layer 14 is exposed. The biodegradable polymer layer 14 gradually degrades with time and thereby the thickness thereof decreases. The biodegradable polymer layer 14 also disappears eventually. The biodegradable polymer layer 14 may dissolve instead of or in addition to degradation. When the biodegradable polymer layer 14 disappears, the drug layer 13 adjoining or in contact with the adhesive portion 12 is exposed. When exposed, the drug layer 13 starts releasing the drug. The thickness of the drug layer 13 gradually decreases as it releases the drug, and the drug layer 13 disappears eventually.

As described above, the drug sustained-release portion 11 gradually releases the drug through at least one of degradation and dissolution of the biodegradable polymer in the biodegradable polymer layer 14. The amount of the drug released from each drug layer 13 and each drug release time are controlled by changing the thickness of each drug layer 13 and/or the components of the drug in each drug layer 13, for example.

Timing of each of the start and the end of releasing the drug from the drug sustained-release portion 11 is controlled by adjusting degradation time or dissolution time of the biodegradable polymer in the biodegradable polymer layer 14. The thickness of the biodegradable polymer layer 14 and/or the types of the biodegradable polymer may be changed to adjust the degradation time or the dissolution time of the biodegradable polymer in the biodegradable polymer layer 14. For example, timing of the drug release from the drug layer 13 adjoining the adhesive portion 12 is delayed when the biodegradable polymer with a small degradation/dissolution rate is used for the biodegradable polymer layer 14 or the thickness of the biodegradable polymer layer 14 is increased. The drug sustained-release portion 11 releases the drug intermittently when the thickness of the biodegradable polymer 14 between the drug layers 13 is large. On the other hand, the drug sustained-release portion 11 releases the drug continuously when the thickness of the biodegradable polymer layer 14 between the drug layers 13 is small.

Note that the drug may be released also from the drug layer 13 adjoining the adhesive portion 12 while the drug is released from the drug layer 13 having the film surface 10a or while the biodegradable polymer in the biodegradable polymer layer 14 degrades or dissolves. This happens because the drug in the drug layer 13 adjoining the adhesive portion 12 may be transferred to the adhesive portion 12 and diffuses inside the adhesive portion 12. The diffused drug is released through the film surface 10b on the adhesive portion 12 side. The drug is transferred through spaces between molecules or molecular chains constituting the adhesive portion 12.

The thickness TH13 of the drug layer 13 is greater than or equal to several nm and less than or equal to several hundreds nm. The thickness TH14 of the biodegradable polymer layer 14 is greater than or equal to several hundreds nm and less than or equal to several μm.

The structure of the drug sustained-release portion 11 is not limited to the example shown in FIG. 1. For example, the drug sustained-release portion 11 maybe composed of three layers: the biodegradable polymer layer 14, the drug layer 13, and the biodegradable polymer layer 14 layered in this order on the adhesive portion 12. In FIG. 1, the film surface 10a of the composite film 10 is the surface of the drug layer 13. Instead, the film surface 10a may be the surface of the biodegradable polymer layer 14. Whether the film surface 10a is the surface of the drug layer 13 or the surface of the biodegradable polymer layer 14 is determined in consideration of storage, usage, ease of handling, the timing of drug release, or the like of the composite film 10.

The number of the drug layers 13 and the number of the biodegradable polymer layers 14 in the drug sustained-release portion 11 are not limited to the example shown in FIG. 1. For example, the drug sustained-release portion 11 may have one drug layer 13 and one biodegradable polymer layer 14. The drug sustained-release portion 11 may have one drug layer 13 and two biodegradable polymer layers 14. The drug sustained-release portion 11 may have three or more drug layers 13 and three or more biodegradable polymer layers 14. Hence, the thickness of the drug sustained-release portion 11 varies with the thickness of each drug layer 13 and the thickness of each biodegradable polymer layer 14, and the number of the drug layers 13 and the number of the biodegradable polymer layers 14.

A well-known adhesive which adheres to a living body may be used as the adhesive portion 12. The adhesive may be a solid or a gel. Examples of the adhesives include gelatine, collagen, hyaluronic acid, methyl cellulose, and carboxymethyl cellulose.

The thickness TH12 of the adhesive portion 12 is greater than or equal to 0.1 μm and less than or equal to 20 μm.

The thickness TH10 of the composite film 10 varies with the thickness TH12 of the adhesive portion 12 and the thickness of the drug sustained-release portion 11.

As described above, the composite film 10 has the drug sustained-release portion 11 with a drug sustained-release function and the adhesive portion 12 with an adhesive function. The composite film 10 is a function separation type film in which different layers have their respective functions. When the drug sustained-release portion 11 is produced from a material non-adhesive to a living body, the composite film 10 may be used as an anti-adhesive film that prevents adhesion of tissue in a living body.

A biologically active or bioactive substance is used as the drug in the drug layer 13. The bioactive substance is at least one of compounds selected from a group consisting of anti-cancer agents, immunosuppressive agents, antibiotics, antirheumatic agents, antithrombotic agents, HMG-CoA (3-Hydroxy-3-methylglutaryl-coenzyme A) reductase inhibitors, ACE (angiotensin converting enzyme) inhibitors, calcium antagonists, lipid-lowering drugs, integrin inhibitors, anti-allergic agents, antioxidants, GPIIbIIIa antagonists, retinoids, flavonoids, carotenoids, lipid improving drugs, DNA synthesis inhibitors, tyrosine kinase inhibitors, antiplatelet drugs, anti-inflammatory drugs, tissue-derived biomaterials, interferon, and NO (carbon monoxide) production promoting substances.

The following is the examples of the biodegradable polymer constituting the biodegradable polymer layer 14.
polylactic acids such as poly(L-lactide)(PLLA) and poly (D, L-lactide) (PLA), and polyglycolic acids, and their copolymers and combinations of any of the above
polyglycolic acids [polyglycolide (PGA)], poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-cotrimethylene carbonate)(PGA/PTMC), poly(D,L-lactide-co-caprolactone) (PLA/PCL), poly(glycolide-co-caprolactone) (PGA/PCL)
polyethylene oxide (PEO)
polydioxanone (PDS)
polypropylene fumarate
poly(ethyl glutamate-co-glutamic acid)
poly(tert-butyloxy-carbonylmethyl glutamate)
polycaprolactone (PCL)
polycaprolactone-co-butylacrylate
polyhydroxybutyrate (PHBT)
polyhydroxybutyrate
poly(phosphazene)
poly(phosphate ester)
poly(amino acid)
poly(hydroxybutyrate)
polydepsipeptide
maleic anhydride copolymer
polyphosphazene
polyiminocarbonate
poly[(97.5% dimethyl-trimethylene carbonate)-co-(2.5% trimethylene carbonate)]
cyanoacrylate
polyethylene oxides
polysaccharides such as methyl cellulose, ethyl cellulose, and acetyl cellulose
the combinations of any of the above, and copolymers

The weight average molecular weight of the above polymers is preferably 5,000 to 1,000,000, and more preferably 10,000 to 500,000.

As shown by a curve B (chain double-dashed line) in FIG. 2 representing an amount of a drug released from a conventional honeycomb-patterned film with time, the drug release amount exceeds a therapeutic range TR at some point of time. After that, the drug release amount drastically decreases and remains below the therapeutic range TR. The conventional honeycomb-patterned film is produced from the biodegradable polymer in which a compound, that is, the drug is dispersed. On the other hand, the composite film, as shown by a curve A (solid line) in FIG. 2, releases the drug with the drug release amount not exceeding the therapeutic range TR, owing to the above-described structure of the drug sustained-release portion 11. The drug release amount of the composite film 10 is maintained within the therapeutic range TR during a period of time longer than that of the conventional film.

In the following embodiments, the descriptions of the elements and their operations similar to those of the composite film 10 of the first embodiment are omitted. The same reference numerals in FIGS. 1 and 3 to 11 denote the same elements.

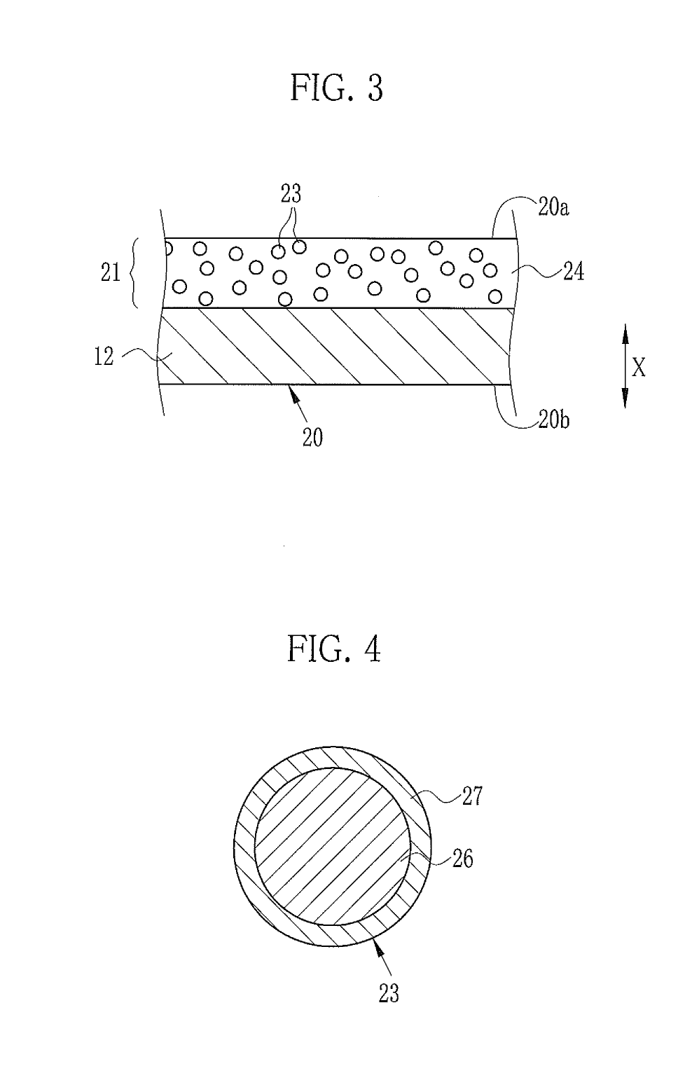

As shown in FIG. 3, a composite film 20 of a second embodiment comprises a drug sustained-release portion 21 and the adhesive portion 12. The drug sustained-release portion 21 releases a drug gradually. The drug sustained-release portion 21 and the adhesive portion 12 are layered on each other in the direction shown by the arrow X. In FIG. 3, a reference numeral 20a denotes the film surface of the drug sustained-release portion 21. A reference numeral 20b denotes the film surface of the adhesive portion 12.

The drug sustained-release portion 21 has drug particles 23 and biodegradable polymer 24. The drug particles 23 are particle-shaped and contain the drug. As shown in FIG. 4, the drug particle 23 is composed of a drug form 26 and an outer coating or a shell 27 that covers the whole surface of the drug form 26. The drug form 26 contains the drug of a predetermined concentration or more. The drug form 26 may consist solely of the drug. The shell 27 is composed of the biodegradable polymer.

The drug particles 23 are embedded and dispersed in the biodegradable polymer 24.

Owing to the above-described structure, the biodegradable polymer 24 dissolves or degrades from the film surface 20a of the drug sustained-release portion 21 of the composite film 20. The drug particles 23 embedded in the biodegradable polymer 24 are exposed as the biodegradable polymer 24 degrades or dissolves. Because a living body has water in it, the shell 27 of the drug particle 23 dissolves or degrades in the water. The drug in the drug form 26 is released to the outside of the composite film 20 when the shell 27 dissolves or degrades in the water. Due to at least one of the dissolution and the degradation of the biodegradable polymer 24, the drug particles 23 are exposed and the drug is released to the outside of the composite film 20. The thickness of the drug sustained-release portion 21 gradually decreases as the biodegradable polymer 24 degrades, and the drug sustained-release portion 21 disappears eventually.

Thus, the drug sustained-release portion 21 gradually releases the drug due to the degradation or dissolution of the biodegradable polymer 24. The drug release amount is controlled by adjusting an amount of the drug particles 23, for example. The drug release amount per unit time is controlled by adjusting the density of the drug particles 23 in the drug sustained-release portion 21 or the size of the drug forms 26, for example. A drug release time is controlled by changing the type of the biodegradable polymer 24 and/or the shell 27, or adjusting the thickness of the shell 27, for example.

The timing of each of the start and the end of releasing the drug from the drug sustained-release portion 21 is controlled by adjusting the degradation time or dissolution time of the biodegradable polymer 24, degradation rate or dissolution rate of the shell 27, or the thickness of the shell 27. The type of the biodegradable polymer 24 may be changed to adjust the degradation time or dissolution time of the biodegradable polymer. The types of the materials of the shell 27 may be changed to adjust the degradation rate and the dissolution rate of the shell 27. For example, timing of the drug release from the drug particles 23 is delayed when the biodegradable polymer 24 with a small degradation/dissolution rate is used, or when the materials of the shell 27 have small water-dissolution rates or small hydrolysis rates, or when the thickness of the shell 27 is large.

Owing to the structure of the drug sustained-release portion 21, the relation between time and the drug release amount of the composite film 20 is also depicted by the slid line A in FIG. 2, similar to that of the composite film 10. The drug is released from the composite film 20, with the drug release amount not exceeding the therapeutic range TR. The drug release amount of the composite film 20 is maintained within the therapeutic range TR in a period of time longer than that of the conventional film.

In this embodiment, the drug particles 23, in each of which the particle-shaped drug form 26 is covered with the shell 27, are dispersed in the biodegradable polymer 24. The drug forms 26 may not be covered with the shells 27 as long as the drug forms 26 are dispersed and embedded in the biodegradable polymer 24. However, it may be preferable to cover the drug forms 26 with the shells 27 in view of producing the composite film 20, for example, dispersing the drug forms 26 in the biodegradable polymer 24, or in view of controlling the timing of releasing the drug.

When the drug sustained-release portion 21 is produced from a material non-adhesive to a living body, the composite film 20 may be used as an anti-adhesive film that prevents adhesion of tissue in a living body.

Note that the biodegradable polymer 24 may be the same as the biodegradable polymer constituting the biodegradable polymer layer 14 of the composite film 10. The components of the drug form 26 may be those described as the components of the drug layer 13 of the composite film 10 shown in FIG. 1.

As described above, instead of or in addition to the biodegradable polymer, a water-soluble polymer or a hydrolyzable polymer may be used for producing the shell 27. Examples of the water-soluble polymers used for producing the shell 27 include gelatine, methyl cellulose, and carboxymethyl cellulose. When the biodegradable polymer is used for producing the shell 27, the above-described substances used for producing the biodegradable polymer layer 14 of the composite film 10 shown in FIG. 1 may be used. The materials of the shell 27 are not limited to the biodegradable polymer, the water-soluble polymer, and the hydrolyzable polymer. The shell 27 may be made from amphiphilic compounds such as phospholipids. The use of the amphiphilic compounds makes dispersion condition of the drug particles 23 in the biodegradable polymer 24 stable. It is preferable that the amphiphilic compounds have water-solubility and hydrolyzability.

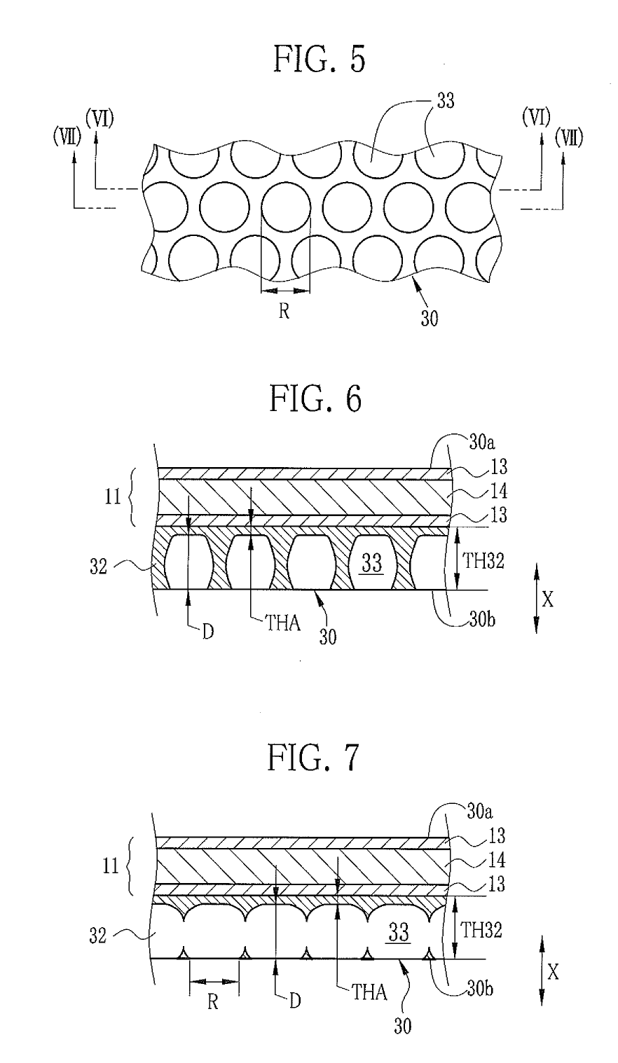

As shown in FIGS. 5 to 7, a composite film 30 of a third embodiment comprises the drug sustained-release portion 11 and an adhesive portion 32. The drug sustained-release portion 11 and the adhesive portion 32 are layered on each other in the thickness direction. In FIGS. 6 and 7, a reference numeral 30a denotes a film surface of the drug sustained-release portion 11. A reference numeral 30b denotes a film surface of the adhesive portion 32. Note that FIG. 5 is a plan view of the composite film 30 viewed from the adhesive portion 32 side (that is, the film surface 30b side).

A plurality of pores 33 are formed on one (that is, the film surface 30b) of the surfaces of the adhesive portion 32. As shown in FIGS. 6 and 7, the pores 33 are not formed through to the opposite side of the film surface 30b of the adhesive portion 32. Namely, the pores 33 are not through holes. The pores 33 are formed as hollows on the film surface 30b.

The pores 33 are formed into an array along the film surface 30b and distributed in two dimensions. The pores 33 are formed such that the adhesive portion 32 has the so-called honeycomb structure. As shown in FIG. 7, the adjoining pores 33 may be interconnected through paths in a direction along the film surface. Alternatively, the adjoining pores 33 may be separated from each other.

The pores 33 have substantially the same shape and size and are arranged regularly. Note that the shape of each opening on the film surface 30b may be circular as shown in FIG. 5 or polygonal, for example, hexagonal, depending on the size and the arrangement of the pores 33.

Diameters R of the openings of the pores 33 on the film surface 30b are substantially the same, in a range greater than or equal to 0.01 μm and less than or equal to 100 μm.

Depths D of the pores 33 are substantially the same, in a range greater than or equal to 0.1 μm and less than or equal to 20 μm.

Here, V1 denotes the volume of the pores 33, and V2 denotes the volume of the adhesive portion 32. The V2 includes the volume V1 of the pores 33. It is preferable to form the pores 33 such that the percentage (%) of the pores 33 calculated by (V1/V2)×100 is greater than or equal to 50% and less than or equal to 90%. Note that the volume V2 represents the volume of the adhesive portion 32 when the film surface 30b is flat (assuming that there are no pores 33).

Owing to the above-described structure of the adhesive portion 32, the pores 33 retain water due to capillary force. Thereby the drug sustained-release portion 11 is adhered to the target site having water, for example, the target site in the living body, through the adhesive portion 32. It is preferable to increase the depth D of the pores 33 relative to the diameter R of the openings in order to increase the adhesive strength, even if the percentage (%) of the pores 33 does not change. The adhesive strength of the adhesive portion 32 which adheres due to the capillary force of the pores 33 is greater than that of the adhesive portion 12 of the composite film 10 shown in FIG. 1. The duration of the adhesive strength of the adhesive portion 32 is also superior to that of the adhesive portion 12.

Note that the thickness TH32 of the adhesive portion 32 is greater than or equal to 0.1 μm and less than or equal to 20 μm. Hence, the thickness THA of a flat portion (a portion between the bottom of the pore 33 and the surface of the adhesive portion 32 opposite to the film surface 30b) of the adhesive portion 32 is greater than 0 μm and less than or equal to several μm.

Owing to the above structure, the relation between time and the drug release amount of the composite film 30 is depicted by the solid line A in FIG. 2, in a manner similar to that of the composite film 10. Namely, the drug is released from the composite film 30 with the drug release amount maintained within the therapeutic range TR in a period of time longer than that of the conventional film. Furthermore, the large adhesive strength of the composite film 30 is maintained. With the use of the composite film 30, the drug is released at the target site and the drug release amount is maintained within the therapeutic range TR securely in a long period of time.

The drug in the drug layer 13 adjoining the adhesive portion 32 is transferred to the adhesive portion 32 and dispersed in the adhesive portion 32. A rate of the drug passing through the adhesive portion 32 increases as the thickness THA of the flat portion of the adhesive portion 32 decreases. Hence, the drug release amount per unit time is controlled by changing the thickness THA of the flat portion of the adhesive portion 32. Owing to the pores 33, the drug release amount per unit time of the adhesive portion 32 is greater than that of the composite film 10 in FIG. 1 or the composite film 20 in FIG. 3.

The adhesive portion 32 may be made from the biodegradable polymer. Thereby the use of the composite film 30 in the living body is extended. Note that, when the adhesive portion 32 is made from the biodegradable polymer, at least one of the degradation rate and the dissolution rate of the biodegradable polymer of the adhesive portion 32 is less than that of the biodegradable polymer of the biodegradable polymer layer 14 of the drug sustained-release portion 11, so that the adhesive portion 32 maintains the adhesive strength until the drug layer 13 of the drug sustained-release portion 11 finishes releasing the drug.

When the drug sustained-release portion 11 of the composite film 30 is produced from a material non-adhesive to a living body, the composite film 30 may be used as an anti-adhesive film that prevents adhesion of tissue in a living body.

When a non-biodegradable polymer is used as a material of the adhesive portion 32, the examples of the non-biodegradable polymer are as follows.
polyolefins such as polyethylene, polypropylene, and polybutylene produced using metallocene catalyst, polybutadiene, polyisobutylene, and their copolymers
vinyl aromatic polymers such as polystyrene
vinyl aromatic copolymers such as styrene-isobutylene-styrene (preferably, TRANSLUTE (registered trademark) produced by Boston Scientific) and styrene-isobutylene copolymers including butadiene-styrene copolymers or other block copolymers
polyethylene vinyl acetate (EVA),
polyvinyl chloride (PVC)
fluorine-based polymers
polyesters
polyamides
polyethers
polyurethanes
polysilicones
polycarbonates
the combinations of any of the above, and copolymers

When the biodegradable polymer is used as a material of the adhesive portion 32, the biodegradable polymer is selected from the same group as that of the biodegradable polymer of the biodegradable polymer layer 14 of the composite film 10 in FIG. 1. As described above, the degradation rate and the dissolution rate of the biodegradable polymer selected are less than those of the biodegradable polymer layer 14 of the drug sustained-release portion 11.

An amphiphilic polymer may be used as a material of the adhesive potion 32 in view of forming the pores 33, which will be described below. The amphiphilic polymer is not particularly limited as long as it is not toxic to the living body. The following examples of the amphiphilic polymers are preferable.
polyethylene glycol-polypropylene glycol block copolymers
amphiphilic polymers having acrylamide polymer as main chains, dodecyl group as hydrophobic side chains, and lactose group or carboxy group as hydrophilic side chains
ion complexes of anionic polymer such as heparin, dextran sulfate, or nucleic acid such as DNA and RNA and long-chain alkyl ammonium salt
amphiphilic polymers having water-soluble protein such as gelatine, collagen, and albumin as hydrophilic group

In particular, amphiphilic polymers containing dodecylacrylamide-ω-carboxyhexylacrylamide are preferable in view of excellence in stabilizing water droplets acting as templates.

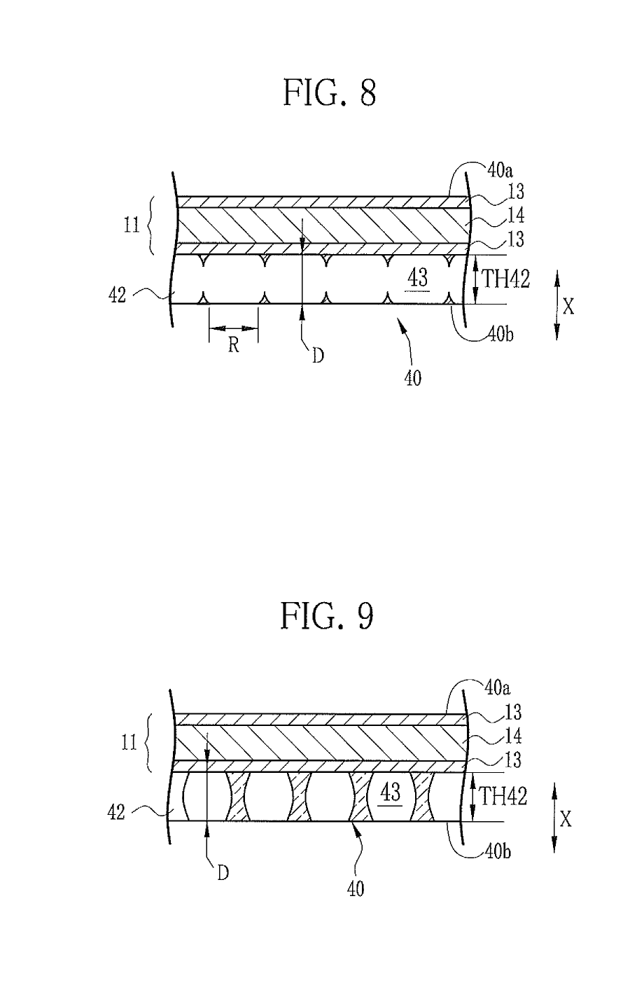

As shown in FIGS. 8 and 9, a composite film 40 of a fourth embodiment comprises the drug sustained-release portion 11 and an adhesive portion 42. The drug sustained-release portion 11 and the adhesive portion 42 are layered on each other in the thickness direction X. In FIGS. 8 and 9, a reference numeral 40a denotes a film surface of the drug sustained-release portion 11. A reference numeral 40b denotes a film surface of the adhesive portion 42. Note that the plan view of the composite film 40 viewed from the film surface 40b side is omitted because it's the same as that shown in FIG. 5.

A plurality of pores 43 are formed in one (the film surface 40b) of the surfaces of the adhesive portion 42. As shown in FIGS. 8 and 9, the pores 43 are through holes penetrating the adhesive portion 42 to the other surface.

The shapes and arrangements of the pores 43, the shapes of the openings of the pores 43 on the film surface 40b, the diameter R of the openings, the depth D of the pores 43, the percentage of the pores, the material of the adhesive portion 42 and the like are similar to those of the pores 33 and the material of the adhesive portion 32 of the composite film 30 and the descriptions thereof are omitted.

Owing to the structure of the adhesive portion 42, the pores 43 retain the water due to the capillary force and thereby the drug sustained-release portion 11 is adhered to the target site having the water, for example, the target site in the living body, through the adhesive portion 42. It is preferable to increase the depth D of the pores 43 relative to the diameter R of the openings in order to increase the adhesive strength, even if the percentage of the pores does not change.

Note that, the thickness TH42 of the adhesive portion 42 is greater than or equal to 0.1 μm and less than or equal to 20 μm.

Owing to the above structure, the relation between time and the drug release amount of the composite film 40 is depicted by the solid line A in FIG. 2, in a manner similar to that of the composite film 10. Namely, the drug is released from a composite film 40, with the drug release amount not exceeding the therapeutic range TR. The drug release amount of the composite film 40 is maintained within the therapeutic range TR in a period of time longer than that of the conventional film. Furthermore, the large adhesive strength of the composite film 40 is maintained. With the use of the composite film 40, the drug is released at the target site and the drug release amount is maintained within the therapeutic range TR securely in a long period of time.

The drug in the drug layer 13 adjoining the adhesive portion 42 is transferred to the adhesive portion 42 and diffuses in the adhesive portion 42. The composite film 40 shown in FIGS. 8 and 9 differs from the composite film 30 shown in FIGS. 5 to 7. The composite film 40 does not have the flat portions (meaning that the pores 43 are through holes) in the adhesive portion 42. Hence, the drug release amount per unit time of the composite film 40 is greater than that of the composite film 30.

When the drug sustained-release portion 11 of the composite film 40 is produced from a material non-adhesive to a living body, the composite film 40 may be used as an anti-adhesive film that prevents adhesion of tissue in a living body.

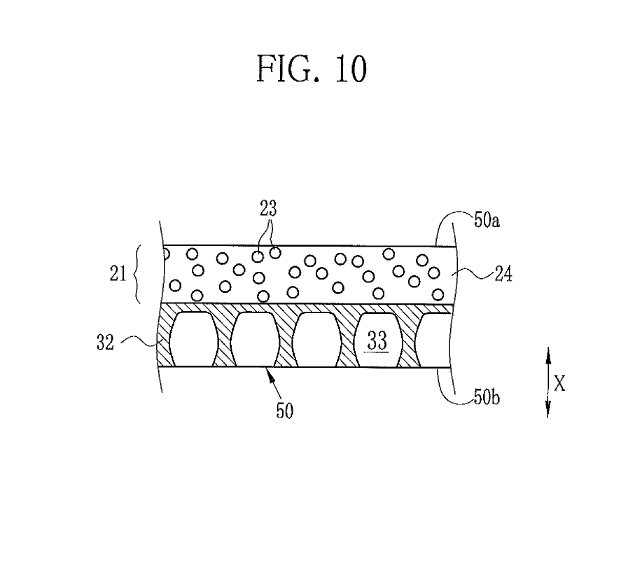

As shown in FIG. 10, a composite film 50 of a fifth embodiment comprises the drug sustained-release portion 21 and the adhesive portion 32. The drug sustained-release portion 21 and the adhesive portion 32 are layered on each other in the thickness direction shown by the arrow X. In FIG. 10, a reference numeral 50a denotes a film surface of the drug sustained-release portion 11. A reference numeral 50b denotes a film surface of the adhesive portion 32. Note that the plan view of the composite film 50 viewed from the film surface 50b side is omitted because it's the same as that in FIG. 5.

Owing to the above structure, the relation between time and the drug release amount of the composite film 50 is depicted by the solid line A in FIG. 2, in a manner similar to that of the composite film 10. Namely, the drug is released from the composite film 50, with the drug release amount not exceeding the therapeutic range TR. The drug release amount of the composite film 50 is maintained within the therapeutic range TR in a period of time longer than that of the conventional film. Furthermore, the large adhesive strength of the composite film 50 is maintained. With the use of the composite film 50, the drug is released at the target site and the drug release amount is maintained within the therapeutic range TR securely in a long period of time.

Note that the adhesive portion 32 of the composite film 50 in FIG. 10 may be replaced with the adhesive portion 42 of the composite film 40 in FIGS. 8-9.

When the drug sustained-release portion 21 of the composite film 50 is produced from a material non-adhesive to a living body, the composite film 50 may be used as an anti-adhesive film that prevents adhesion of tissue in a living body.

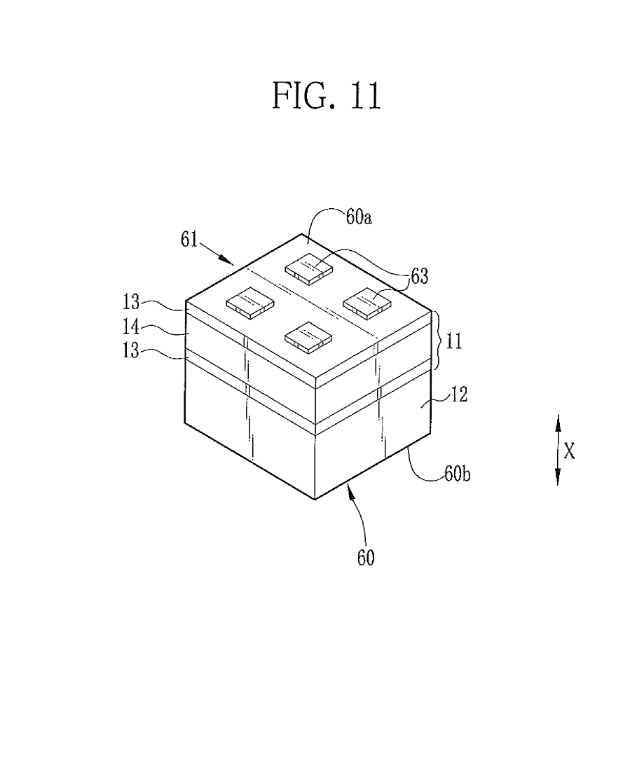

As shown in FIG. 11, a composite film 60 of a sixth embodiment comprises a drug sustained-release portion 61 and the adhesive portion 12. The adhesive portion 12 is the same as that in the composite film 10 shown in FIG. 1. Instead of the adhesive portion 12, the adhesive portion 32, which is the same as that in the composite film 30 shown in FIGS. 5-7, or the adhesive portion 42, which is the same as that in the composite film 40 in FIGS. 8-9, may be used.

The drug sustained-release portion 61 and the adhesive portion 12 are layered on each other in the thickness direction X. In FIG. 11, a reference numeral 60a denotes a film surface of the drug sustained-release portion 61. A reference numeral 60b denotes a film surface of the adhesive portion 12. In FIG. 11, the thicknesses of the drug sustained-release portion 61 and the adhesive portion 32 are exaggerated relative to the area of the composite film 60 in the direction of the normal to the film surfaces 60a and 60b.

The drug sustained-release portion 61 has the structure in which diffusion inhibitors 63 are added to the drug sustained-release portion 11 of the composite film 10 shown in FIG. 1. The diffusion inhibitors 63 are provided on the film surface 60a of the drug layer 13. In this embodiment, each diffusion inhibitor 63 has a rectangular shape, but may have a different shape, for example, a circular shape.

The diffusion inhibitor 63 is formed of the water-soluble polymer, the biodegradable polymer, or the like. The diffusion inhibitor 63 delays or slows down diffusion of the drug from the film surface 60a of the drug layer 13. Thereby the drug release amount is inhibited to a small extent. The diffusion inhibitor 63 reduces the drug release amount at the initial stage (at the beginning of the use of the composite film 60). The number of the diffusion inhibitors 63 and the area of each diffusion inhibitor 63 in the direction of the normal to the film surface 60a may be determined in accordance with the intended or required drug release amount.

When the drug sustained-release portion 61 of the composite film 60 is produced from a material non-adhesive to a living body, the composite film 60 may be used as an anti-adhesive film that prevents adhesion of tissue in a living body.

Hereinafter, methods for producing the composite films 10, 20, 30, 40, 50, and 60 are described. The composite film 10 shown in FIG. 1 is produced using a solution casting method well-known as one of film producing methods or a combination of the solution casting method and a coating method. The solution casting method may be a co-casting method or a sequential casting method.

When the composite film 10 is produced using the solution casting method, an adhesive portion solution for forming the adhesive potion 12, a drug layer solution for forming the drug layer 13, and a biodegradable polymer solution for forming the biodegradable polymer layer 14 are prepared. In the co-casting method, the adhesive portion solution, the drug layer solution, and the biodegradable polymer solution are cast simultaneously on a support and dried. In the sequential casting method, the adhesive portion solution, the drug layer solution, the biodegradable polymer solution, and the drug layer solution are cast sequentially in this order on a support and dried. Note that the solutions may be cast on the support in the following order: the drug layer solution, the biodegradable polymer solution, the drug layer solution, and the adhesive portion solution. When the solution casting method is used, the solutions may be dried on the support, or dried partly on the support and then completely dried after peeling.

Alternatively, a film member for the adhesive portion 12 (that is, the film member which becomes the adhesive portion 12) may be produced using the solution casting method. Then, the drug layer solution, the biodegradable polymer solution, and the drug layer solution may be applied in this order on the film member for the adhesive portion 12 and dried to produce the composite film 10.

The composite film 20 shown in FIG. 3 is produced by the well-known solution casting method or the combination of the solution casting method and the coating method, in a manner similar to the production of the composite film 10 shown in FIG. 1.

When the solution casting method is used to produce the composite film 20, the adhesive portion solution for forming the adhesive portion 12 and the drug sustained-release portion solution for forming the drug sustained-release portion 21 are prepared. The adhesive portion solution and the drug sustained-release portion solution are cast simultaneously or sequentially and dried. Thereby the composite film 20 is produced.

When the composite film 20 is produced by the combination of the solution casting method and the coating method, first, a film member for the adhesive portion 12 is produced by the solution casting method. Then the drug sustained-release portion solution is cast on the film member for the adhesive portion 12 and dried.

The drug sustained-release portion solution for forming the drug sustained-release portion 21 is prepared using the following method, for example. First, the drug particles 23 are produced using a well-known microcapsule production method. For example, the drug particles 23, in each of which the drug form 26 is coated with the shell 27, are produced using the following microcapsulation process. First, the drug is dissolved in water to prepare a drug solution of a predetermined concentration. Gelatine or the like is dissolved or suspended in the drug solution. The solution or the suspension is added to a solution containing the biodegradable polymer, and emulsified using a mixer, for example, a propeller-type mixer. Thereby an emulsion containing the drug agent is prepared. A solvent is evaporated from the emulsion using a drying-in-water method, a phase separation method, a spray drying method, or the like. Thereby the drug, the gelatine, and the like are covered by the biodegradable polymer and microcapsulated. Thus the drug particles 23 are obtained. The drug particles 23 are dispersed in a liquid containing the biodegradable polymer 24. Thus the drug sustained-release portion solution is obtained.

For the production of the composite film 30 shown in FIGS. 5-7, the composite film 40 shown in FIGS. 8-9, and the composite film 50 shown in FIG. 10, a film member for the adhesive portion 32 or 42 is produced. Then the drug sustained-release portion 11 or 21 is formed on the film member for the adhesive portion 32 or 42. A method for forming the drug sustained-release portion 11 or 21 on the film member for the adhesive portion 32 or 42 is similar to the method for forming the drug sustained-release portion 11 or 21 of the composite film 10 or 20 through coating.

Film members for the adhesive portions 32 and 42 are produced by a well-known condensation method. Thereby the adhesive portions 32 and 42 securely retain the water in their respective pores 33 and 43 and have the adhesive strength due to the capillary force.

In the condensation method, the adhesive portion solution for forming the adhesive portion 32 or 42 is cast on the support to form a casting film. Ambience moisture of the casting film is condensed on the casting film before the casting film dries up. The casting film may be cooled from the backside to condense the ambient moisture. Water droplets may be densely arranged by feeding a gas in a predetermined direction or inclining the casting film, with the water droplets formed on the film surface. When the water droplets are formed and get into the solution casting film, the solution casting film is dried by forced drying to evaporate the solvent from the adhesive portion solution.

The film member for the adhesive portion 42 with the penetrated pores (through-holes) in the depth direction or the film member for the adhesive portion 32 with the bottomed pores (hollows) is formed by controlling the depth of the water droplets in the adhesive portion solution.

The diameter R of each opening is controlled by adjusting the growth of the water droplets before the forced evaporation of the solvent.

For the production of the composite film 60 shown in FIG. 11, a diffusion inhibitor solution for forming the diffusion inhibitor 63 is cast on the composite film 10 shown in FIG. 1, for example, and dried.

Various changes and modifications are possible in the present invention and may be understood to be within the present invention.

What is claimed is:

1. A composite film comprising:
a drug sustained-release portion containing a biodegradable polymer and a drug form, the drug form containing a drug of a predetermined concentration or more, the drug sustained-release portion releasing the drug from the drug form through at least one of degradation and dissolution of the biodegradable polymer; and
an adhesive portion through which the drug sustained-release portion is adhered to a target site, the adhesive portion being layered on the drug sustained-release portion.

2. The composite film of claim 1, wherein the drug sustained-release portion has the film-like drug form and the film-like biodegradable polymer on the film-like drug form.

3. The composite film of claim 1, wherein the drug sustained-release portion has the particle-like drug forms and the biodegradable polymer in which the particle-like drug forms are embedded.

4. The composite film of claim 1, wherein a plurality of pores are formed into an array on one surface of the adhesive portion, opposite to the other surface in contact with the drug sustained-release portion, and the pores of the adhesive portion retain water of the target site due to capillary force and thereby the drug sustained-release portion is adhered to the target site through the adhesive portion.

5. The composite film of claim 4, wherein the adhesive portion is composed of a biodegradable polymer in which at least one of a degradation rate and a dissolution rate is smaller than that of the biodegradable polymer of the drug sustained-release portion.
